(19) United States
(12) Patent Application Publication
(10) Pub. No.: US 2013/0195962 A1
(43) Pub. Date: Aug. 1, 2013

(54) LIPID MEMBRANE STRUCTURE

(71) Applicants: Hideyoshi Harashima, Hokkaido (JP); Hiroto Hatakeyama, Hokkaido (JP); Ikramy Khalil, Assiut (EG); Kazuhiro Takara, Osaka (JP)

(72) Inventors: Hideyoshi Harashima, Hokkaido (JP); Hiroto Hatakeyama, Hokkaido (JP); Ikramy Khalil, Assiut (EG); Kazuhiro Takara, Osaka (JP)

(73) Assignee: NATIONAL UNIVERSITY CORPORATION HOKKAIDO UNIVERSITY, Hokkaido (JP)

(21) Appl. No.: 13/642,699

(22) PCT Filed: Feb. 23, 2011
(86) PCT No.: PCT/JP2011/053963

§ 371 Date: Apr. 2, 2013

(30) Foreign Application Priority Data
Apr. 28, 2010 (JP) 2010-103333

(51) Int. Cl. A61K 9/127

(52) U.S. Cl. CPC A61K 9/127
USPC 424/450; 514/44 R; 514/34

(57) ABSTRACT

A lipid membrane structure for delivering a substance to a target cell having satisfactory cell permeability, selectivity for target cell, and in vivo stability, wherein lipid membrane is modified with (a) a polyalkylene glycol bound with a target cell-selective ligand; and (b) a polypeptide comprising a plurality of arginine residues.

### TECHNICAL FIELD

The present invention relates to a lipid membrane structure that can efficiently deliver a substance such as a nucleic acid into a target cell. More specifically, the present invention relates to a lipid membrane structure that can efficiently deliver a nucleic acid and the like into a target cell, and has superior cell permeability, selectivity for target cell, and in vivo stability.

### BACKGROUND ART

For lipid membrane structures such as liposomes used as a drug delivery system (DDS), various characteristic features are required, such as drug retaining ability, in vivo stability, selectivity for a specific cell (target cell) to which a medicament is to be delivered, permeability into target cell, and ability to release a medicament in a target cell. In particular, for lipid membrane structures for delivering a medicament or a nucleic acid into cytoplasm or nucleus of a target cell, superior permeability into cells, in particular, specific organelles in cells such as nuclei, is required, as well as target cell selectivity.

The inventors of the present invention previously found that by modifying surface of a lipid membrane structure with a peptide comprising contiguous arginine residues, such as octaarginine (R8), cell permeability, especially nucleus permeability, of the lipid membrane structure was successfully improved (International Patent Publication WO2005/032593; Journal of Controlled Release, 98, pp. 317-323, 2004). However, the modification with R8 does not give specific cell-selective permeability. Further, a lipid membrane structure modified with R8 is easily and promptly eliminated from the living body due to the high cationic property of R8, and therefore it is considered to be difficult to selectively deliver a lipid membrane structure modified with R8 to a target cell.

As a method for enhancing in vivo stability of lipid membrane structures, especially stability in blood, methods of modifying surface of a lipid membrane structure with a polyalkylene glycol, typically polyethylene glycol (PEG), are widely known (described in, for example, Japanese Patent Unexamined Publication (KOKAI) Nos. 1-249717, 2-149512, 4-346918, 2004-10481, and the like). These methods are based on the fact that if a hydrated layer formed with PEG covers microparticle carriers such as lipid membrane structures, opsonization such as adsorption of serum proteins is suppressed, and as a result, phagocytosis by macrophages and uptake by reticuloendothelial tissues can be avoided.

However, when surface of a lipid membrane structure modified with R8 is further modified with PEG in order to enhance in vivo stability of the lipid membrane structure, there arises a problem that the cell permeability imparted by the modification with R8 is lost. Specifically, if a lipid membrane structure is simultaneously modified with R8 and PEG, there arises a confliction, i.e., in vivo stability of the lipid membrane structure is improved by the modification with PEG, whist cell permeability is degraded (also called as “PEG dilemma”).

As an attempt for solving this PEG dilemma, the inventors of the present invention created a phospholipid derivative in which a peptide containing a substrate peptide that can serve as a substrate of a matrix metalloprotease is further placed between a PEG residue and a phospholipid residue, and proposed a lipid membrane structure containing this lipid derivative (Japanese Patent Unexamined Publication (KOKAI) No. 2007-099750). Since this lipid membrane structure has a characteristic feature that the peptide moiety is cleaved by a matrix metalloprotease to allow resulting release of PEG, the structure also has a characteristic feature that it is stable in blood due to the presence of the modified moiety, but stability as the whole lipid membrane structure is reduced when it locates near malignant tumor cells secreting a matrix metalloprotease because the modified moiety is released. On the basis of the aforementioned characteristic, an antitumor agent or a nucleic acid retained by the lipid membrane structure is released near the outside of malignant tumor cells, or the lipid membrane structure of which modified moiety has been released is efficiently taken up into malignant tumor cells, and therefore the medicament or nucleic acid can be efficiently introduced into the malignant tumor cells.

However, although the technique described in Japanese Patent Unexamined Publication (KOKAI) No. 2007-099750 mentioned above is effective as a DDS means for malignant tumor cells secreting a matrix metalloprotease, almost no effectiveness thereof as a DDS means for other target cells, especially target cells not secreting matrix metalloprotease or secreting only a small amount of matrix metalloprotease, can be expected, and it also does not improve selectivity for cells other than malignant tumor cells.

As a method for enhancing selectivity of lipid membrane structures for target cells, there is known a method of modifying surface of a lipid membrane structure with a ligand that can selectively bind to a biological substance specifically expressed in specific cells, such as receptors existing on cell membrane surfaces. In this method, in order to simultaneously attain improvement of in vivo stability of lipid membrane structures and selectivity for target cells, the ligand is generally often placed on a surface of a lipid membrane structure via PEG. However, although selectivity for target cells of the aforementioned lipid membrane structure is improved by the modification with a specific ligand, it has a problem that, since the lipid membrane structure is taken up into cells by saturable receptor-mediated endocytosis, cell permeation of the lipid membrane structure is saturatedly limited, and improvement in uptake of a medicament or the like cannot be obtained to an expected extent. As described above, at present, any lipid membrane structures simultaneously satisfying cell permeability, selectivity for target cell, and in vivo stability have not been provided.

### PRIOR ART REFERENCES

### Patent Documents

Patent document 1: International Patent Publication WO2005/032593
Patent document 2: Japanese Patent Unexamined Publication (KOKAI) No. 1-249717
Patent document 3: Japanese Patent Unexamined Publication (KOKAI) No. 2-149512
Patent document 4: Japanese Patent Unexamined Publication (KOKAI) No. 4-346918
Patent document 5: Japanese Patent Unexamined Publication (KOKAI) No. 2004-10481
Patent document 6: Japanese Patent Unexamined Publication (KOKAI) No. 2007-099750

### Non-Patent Document

Non-patent document 1: Journal of Controlled Release, 98, pp. 317-323, 2004

### SUMMARY OF THE INVENTION

### Object to be Achieved by the Invention

An object of the present invention is to provide a lipid membrane structure that satisfies satisfactory cell permeability, selectivity for target cell, and in vivo stability.

### Means for Achieving the Object

The inventors of the present invention conducted various researches to achieve the aforementioned object, and investigated detailed influence of surface modification of a lipid membrane structure with (a) a cellular uptake-promoting peptide having several contiguous arginine residues (this peptide is henceforth also referred to as “polyarginine” or RX) such as R8, and (b) a polyalkylene glycol bound with a target cell-selective ligand (ligand-PAG). As a result, it was revealed that when a lipid membrane structure was modified with RX and PAG, the cellular uptake function of polyarginine was inhibited by PAG, and uptake amount of the lipid membrane structure into cells and gene expression amount of a gene encapsulated in the lipid membrane structure are reduced as compared with non-PAG-modified lipid membrane structures, and that a non-RX-modified lipid membrane structure modified only with ligand-PAG did not give increase of uptake amount of the lipid membrane structure into cells expressing a target biological substance for the ligand. Whilst, it was surprisingly found that a lipid membrane structure modified with both RX and ligand-PAG provided increase of cellular uptake amount and marked increase of activity for gene expression from a nucleic acid encapsulated therein.

As explained above, it was already known that if a lipid membrane structure modified with a polyarginine and having a cellular uptake function was modified with a polyalkylene glycol, the function of the polyarginine was inhibited. However, it was found that if surface of a lipid membrane structure was modified with a polyalkylene glycol bound with a target cell-selective ligand and a polyarginine, the inhibition of the function of polyarginine by polyalkylene glycol was effectively eliminated, and a lipid membrane structure having satisfactory in vivo stability, selectivity for target cell provided by the ligand, and cell permeability was successfully provided. The present invention was accomplished on the basis of the aforementioned finding.

The present invention thus provides a lipid membrane structure for delivering a substance to a target cell, wherein lipid membrane is modified with the following (a) and (b):

(a) a polyalkylene glycol bound with a target cell-selective ligand; and
(b) a polypeptide comprising a plurality of arginine residues.

According to preferred embodiments of the aforementioned lipid membrane structure, there are provided the aforementioned lipid membrane structure, wherein the lipid membrane structure is a liposome; the aforementioned lipid membrane structure, wherein the target cell-selective ligand is a ligand that can specifically bind to a receptor expressed outside a cell membrane of the target cell; the aforementioned lipid membrane structure, wherein the target cell-selective ligand is bound to a tip end portion of the aforementioned polyalkylene glycol; the aforementioned lipid membrane structure, wherein (a) the polyalkylene glycol and (b) the polypeptide are modified with a hydrophobic group, preferably stearyl group, cholesteryl group, or the like, and the hydrophobic group is inserted into the lipid membrane; the aforementioned lipid membrane structure, wherein (b) the polypeptide is a polypeptide containing 4 to 20 contiguous arginine residues, preferably a polypeptide consisting only of 4 to 20 contiguous arginine residues, more preferably octaarginine; the aforementioned lipid membrane structure, wherein (a) the polyalkylene glycol is a polyethylene glycol (PEG); and the aforementioned lipid membrane structure, wherein ratio of cationic lipids to the total lipids constituting a lipid bilayer is 0 to 40% (molar ratio).

Further, as another embodiment, there is provided any of the aforementioned lipid membrane structures, wherein the substance to be delivered is encapsulated in the inside of the lipid membrane structure. According to preferred embodiments of this invention, there are provided any of the aforementioned lipid membrane structures, wherein the substance to be delivered is a nucleic acid, for example, a functional nucleic acid such as a nucleic acid containing a gene or an siRNA; any of the aforementioned lipid membrane structures, wherein the lipid membrane structure is a multifunctional envelope-type nano device (MEND); and any of the aforementioned lipid membrane structures, wherein a nucleic acid and a cationic polymer, preferably protamine, are encapsulated in the inside of the lipid membrane structure.

There is further provided any of the aforementioned lipid membrane structures, wherein an antitumor agent is encapsulated in the inside of the lipid membrane structure. According to preferred embodiments of this invention, there are provided the aforementioned lipid membrane structure, wherein the antitumor agent is doxorubicin; the aforementioned lipid membrane structure, wherein the target cell-selective ligand is a ligand peptide; the aforementioned lipid membrane structure, which is in the form of a liposome; and the aforementioned lipid membrane structure, which has a particle size in the range of about 200 to 400 nm, preferably a particle size of about 300 nm.

The present invention also provides the aforementioned lipid membrane structure, which is used for gene expression in vivo in a cell of a mammal including human; the aforementioned lipid membrane structure, which is used for a gene therapy of a mammal including human; the aforementioned lipid membrane structure, which is used for a therapeutic treatment of a malignant tumor in a mammal including human; and a pharmaceutical composition comprising the aforementioned lipid membrane structure as an active ingredient, preferably such a pharmaceutical composition containing a nucleic acid or an antitumor agent as a substance to be delivered.

From another aspect of the present invention, there is provided a method for delivering a substance to a cell of a mammal including human in vivo, which comprises the step of administering a lipid membrane structure encapsulating a substance to be delivered to the mammal, wherein lipid membrane of the lipid membrane structure is modified with (a) a polyalkylene glycol bound with a target cell-selective ligand; and (b) a polypeptide comprising a plurality of arginine residues. Examples of the substance to be delivered include a medicament for prophylactic and/or therapeutic treatment of a disease, preferably a nucleic acid for a gene therapy, an antitumor agent, and the like.

From still another aspect of the present invention, there is provided a method for expressing a gene in an in vivo cell of a mammal including human, which comprises the step of administering a lipid membrane structure encapsulating a nucleic acid containing the gene as a substance to be delivered to the mammal, wherein lipid membrane of the lipid membrane structure is modified with (a) a polyalkylene glycol bound with a target cell-selective ligand; and (b) a polypeptide comprising a plurality of arginine residues. The aforementioned lipid membrane structure encapsulating a cationic polymer, for example, protamine, together with the nucleic acid can be preferably used. Furthermore, there is provided the aforementioned method, which is used for a gene therapy.

The present invention also provides a method for prophylactic and/or therapeutic treatment of a disease of a mammal including human, which comprises the step of administering a prophylactically and/or therapeutically effective amount of a lipid membrane structure encapsulating a substance to be delivered to the mammal, wherein lipid membrane of the lipid membrane structure is modified with (a) a polyalkylene glycol bound with a target cell-selective ligand; and (b) a polypeptide comprising plural arginine residues. As preferred embodiments of this invention, there are provided the aforementioned method, wherein the substance to be delivered is a medicament for prophylactic and/or therapeutic treatment of the disease; and the aforementioned method, wherein the substance to be delivered is a nucleic acid containing a gene or an antitumor agent.

### Effect of the Invention

If a lipid membrane structure is modified with both of polyarginine and PEG, there arises a conflicting problem, that is, although in vivo stability of the lipid membrane structure is improved by the modification with PEG, cell permeability is degraded (PEG dilemma). However, the lipid membrane structure of the present invention simultaneously achieves superior in vivo stability, selectivity for target cell provided by a ligand, and cell permeability, and accordingly, the structure is extremely useful for uses as, for example, a lipid membrane structure for delivering a nucleic acid containing a gene into a cell and expressing it, or a lipid membrane structure for selectively delivering an antitumor agent to a malignant tumor.

### BRIEF DESCRIPTION OF THE DRAWINGS

FIG. 1 Graphs showing cellular uptake-promoting effect of liposomes of which surfaces were modified with PEG bound with a peptide ligand and R4.

FIG. 2 A graph showing cellular uptake-promoting effect of liposome of which surfaces were modified with PEG bound with a peptide ligand and R4 by way of comparison with other liposomes.

FIG. 3 A graph showing results indicating reduction of gene expression activity of pDNA core-encapsulated liposomes modified with octaarginine (R8) depending on modification amount with PEG (comparative example).

FIG. 4 A graph showing gene expression activity of liposomes modified with Tf bound to PEG and octaarginine.

FIG. 5 A graph showing results of investigation of antitumor effect of liposomes encapsulating doxorubicin and surface-modified with PEG bound with a peptide ligand and R4. In the graph, “Small” indicates results obtained by a liposome having a particle size of 100 nm (dose: 1.5 mg/kg) and “Large” by a liposome having a particle size of 300 nm (dose: 1.0 mg/kg or 6.0 mg/kg).

FIG. 6 A graph showing results of investigation of antitumor effect of liposomes surface-modified with PEG bound with a peptide ligand and R4 and encapsulating doxorubicin at a dose of 6.0 mg/kg. In the graph, Large means a liposome having a particle size of 300 nm, and Large PEG for results by modification only with PEG, Large NGR-PEG for modification only with PEG bound with a peptide ligand (NGR), Large R4/PEG for modification with R4 and PEG, Large Dual for modification with PEG bound with a peptide ligand and R4. Doxil indicates results obtained by commercially available liposome encapsulating doxorubicin and having a particle size of 100 nm.

FIG. 7 A photograph showing result of in vivo investigation of permeation of PEG-modified liposomes having a particle size of 300 nm (PEG concentration: 10 mol %, EPC/Chol=7:3, lipids were labeled with 1 mol % rhodamine) used at a dose of 0.5 μmol lipids/250 μl into vascular endothelial cells. The liposomes are indicated in red, the endothelial cells are indicated in green, and nuclei are indicated in blue.

FIG. 8 A photograph showing result of investigation of permeation in vivo of liposomes modified with ligand peptide (NGR)-bound PEG and having a particle size of 300 nm (PEG concentration: 10 mol %, EPC/Chol=7:3, lipids were labeled with 1 mol % rhodamine) used at a dose of 0.5 μmol lipids/250 μl into vascular endothelial cells. The liposomes are indicated in red, the endothelial cells are indicated in green, and nuclei are indicated in blue.

FIG. 9 A photograph showing result of i investigation of permeation n vivo of liposomes modified with PEG and R4 and having a particle size of 300 nm (PEG concentration: 10 mol %, STR-R4 concentration: 2.5 mol %, EPC/Chol=7:3, lipids were labeled with 1 mol % rhodamine) used at a dose of 0.5 μmol lipids/250 μl into vascular endothelial cells. The liposomes are indicated in red, the endothelial cells are indicated in green, and nuclei are indicated in blue.

FIG. 10 A photograph showing result of investigation of permeation in vivo of liposomes modified with ligand peptide (NGR)-bound PEG and R4 and having a particle size of 300 nm (PEG concentration: 10 mol %, STR-R4 concentration: 2.5 mol %, EPC/Chol=7:3, lipids were labeled with 1 mol % rhodamine) used at a dose of 0.5 μmol lipids/250 μl into vascular endothelial cells. The liposomes are indicated in red, the endothelial cells are indicated in green, and nuclei are indicated in blue.

### MODES FOR CARRYING OUT THE INVENTION

Examples of lipids constituting the lipid membrane structure of the present invention include, for example, phospholipids, glycolipids, sterols, saturated or unsaturated fatty acids, and the like.

Examples of the phospholipids and phospholipid derivatives include, for example, phosphatidylethanolamine, phosphatidylcholine, phosphatidylserine, phosphatidylinositol, phosphatidylglycerol, cardiolipin, sphingomyelin, ceramide phosphorylethanolamine, ceramide phosphorylglycerol, ceramide phosphorylglycerol phosphate, 1,2-dimyristoyl-1,2-deoxyphosphatidylcholine, plasmalogen, phosphatidic acid, and the like, and one or more kinds of these can used independently or in combination. Although the fatty acid residues of these phospholipids are not particularly limited, examples include saturated or unsaturated aliphatic acid residues having 12 to 20 carbon atoms, and specific examples include, for example, acyl groups derived from such a fatty acid as lauric acid, myristic acid, palmitic acid, stearic acid, oleic acid, and linoleic acid. Further, a phospholipid derived from a natural product such as egg yolk lecithin and soybean lecithin can also be used.

Examples of the glycolipids include glyceroglycolipids (for example, sulfoxyribosyl glyceride, diglycosyl diglyceride, digalactosyl diglyceride, galactosyl diglyceride, and glycosyl diglyceride), sphingoglycolipids (for example, galactosyl cerebroside, lactosyl cerebroside and ganglioside), and the like.

Examples of the sterols include animal-derived sterols (for example, cholesterol, cholesterol succinate, lanosterol, dihydrolanosterol, desmosterol and dihydrocholesterol), plant-derived sterols (phytosterol) (for example, stigmasterol, sitosterol, campesterol and brassicasterol), microorganism-derived sterols (for example, thymosterol and ergosterol), and the like.

Examples of the saturated or unsaturated fatty acids include saturated or unsaturated fatty acids having 12 to 20 carbon atoms, such as palmitic acid, oleic acid, stearic acid, arachidonic acid, and myristic acid.

Form of the lipid membrane structure is not particularly limited, and examples of the form in which lipid membrane structures are dispersed in an aqueous solvent include unilamella liposomes, multi-lamella liposomes, 0/W type emulsions, W/O/W type emulsions, spherical micelles, fibrous micelles, layered structures of irregular shapes and the like. Examples of preferred form of the lipid membrane structure of the present invention include liposomes. Although liposomes may be explained hereafter as a preferred embodiment of the lipid membrane structure of the present invention, the lipid membrane structure of the present invention is not limited to liposomes.

The lipid membrane structure of the present invention is characterized in that the lipid membrane is modified with (a) a polyalkylene glycol bound with a target cell-selective ligand at a tip end portion thereof, and (b) a polypeptide comprising a plurality of arginine residues, and is a lipid membrane structure used for delivering a substance to a target cell.

Means for modifying surfaces of lipid membrane structures with a polyalkylene glycol to enhance blood retainability of liposomes are described in, for example, Japanese Patent Unexamined Publication (KOKAI) Nos. 1-249717, 2-149512, 4-346918, 2004-10481, and the like. As the polyalkylene glycol, for example, polyethylene glycol, polypropylene glycol, polytetramethylene glycol, polyhexamethylene glycol, and the like can be used. Molecular weight of the polyalkylene glycol is, for example, about 300 to 10,000, preferably about 500 to 10,000, more preferably about 1,000 to 5,000.

In the lipid membrane structure of the present invention, the aforementioned polyalkylene glycol bound with a target cell-selective ligand can be used. When the surface modification with polyalkylene glycol is performed, a target cell-selective ligand can be bound to all or a part of polyalkylene glycol molecules to be used. Although position of the polyalkylene glycol at which the target cell-selective ligand is bound is not particularly limited, it is preferably a tip end portion of the polyalkylene glycol.

The “tip end portion” of the polyalkylene glycol referred to in the specification means a portion around an end of the polyalkylene glycol, which is not the end bound to the lipid membrane structure, among the both ends of the polyalkylene glycol. In general, the target cell-selective ligand can be bound to a tip end portion of a linear polyalkylene glycol or a tip end portion of a main chain or side chain of a branched polyalkylene glycol. Two or more target cell-selective ligand molecules may be bound to one polyalkylene glycol molecule.

In the specification, the “target cell” means a cell serving as a target to which a substance such as a nucleic acid and a medicament is delivered by using the lipid membrane structure of the present invention, and a cell having a receptor to which the target cell-selective ligand can specifically bind may be used as the target cell. Type of the target cell is not particularly limited, and an appropriate cell can be used as the target cell depending on type of the substance to be delivered, purpose of the delivery of the substance, and the like. For example, the target cell may be a cell that constitutes a tissue or organ, or may be a cell that independently exists such as a leukemic cell. The target cell may also be a cell forming a tumor in a normal tissue such as a solid tumor cell, or a cell infiltrating into a lymphoid tissue or other tissues.

The “ligand” referred to in the specification means a substance having an ability to bind to a receptor, and typically, a substance that can specifically bind to a receptor can be used. The receptor is a substance that can bind a ligand, and generally means a substance having an action for starting a certain reaction by binding with a ligand. In the specification, the terms “ligand” and “receptor” are used to refer to partners that can bind with each other, preferably partners that can specifically bind with each other, and they should not be construed to be limited to those of which binding induces a certain biological reaction. For example, if an antibody is used as the ligand, and an antigen is used as the receptor, binding of them may not induce any biological reaction, but these are also encompassed in the scopes of the aforementioned terms. Therefore, in the specification, the ligand includes neuromessengers, hormones, cell growth factors, enzyme substrates, and the like, as well as antibodies and fragments thereof, proteins, and the like, and the receptor includes receptors generally consisting of a protein, as well as enzymes, and low molecular weight substances that can serve as antigen (lipid compounds, sugar compounds, polypeptides, oligopeptides, and the like).

More specifically, as the ligand, besides low molecular weight organic compounds, for example, dipeptides, tripeptides, oligopeptides, polypeptides, proteins, and the like may be used. As oligopeptides, for example, an oligopeptide comprising about 4 to 20 amino acid residues may be used, and as the polypeptides, a polypeptide comprising more than 20 amino acid residues can be used. For example, as the ligand, an antibody that can specifically bind with a cell surface antigen, preferably a monoclonal antibody or a fragment thereof (for example, Fab fragment, F(ab′)₂ fragment, Fab′ fragment and the like) may also be used. In such a case, besides low molecular weight compounds existing on cell surfaces (for example, sugar compounds and lipid compounds), various antigens such as oligopeptides and proteins may serve as the receptor, and in the specification, the term of receptor must be construed in its broadest sense including the aforementioned antigens, and the like. As the receptor, receptors existing on cell membrane surface of the target cell can be preferably used, and as the target cell-selective ligand, a low molecular weight substance that can specifically bind with the receptor existing on the cell membrane surface of the target cell, for example, a peptide compound that can specifically bind with the receptor existing on the cell membrane surface of the target cell (ligand peptide) and the like can be used.

The surface modification of the lipid membrane structure with a polyalkylene glycol can be easily performed by constructing the lipid membrane structure using, for example, a polyalkylene glycol-modified lipid as a lipid membrane-constituting lipid. For example, when the modification with a polyethylene glycol is performed, stearylated polyethylene glycols (for example, PEG45 stearate (STR-PEG45) and the like) can be used. In addition, polyethylene glycol derivatives, such as N-{carbonyl-methoxypolyethylene glycol 2000}-1,2-dipalmitoyl-sn-glycero-3-phosphoethanolamine, n-{carbonyl-methoxypolyethylene glycol 5000}-1,2-dipalmitoyl-sn-glycero-3-phosphoethanolamine, N-{carbonyl-methoxypolyethylene glycol 750}-1,2-distearoyl-sn-glycero-3-phosphoethanolamine, N-{carbonyl-methoxypolyethylene glycol 2000}-1,2-distearoyl-sn-glycero-3-phosphoethanolamine, and N-{carbonyl-methoxypolyethylene glycol 5000}-1,2-distearoyl-sn-glycero-3-phosphoethanolamine, can also be used. However, the polyalkylene glycolated lipid is not limited to these examples.

Although means for binding the target cell-selective ligand to the polyalkylene glycol is not particularly limited, for example, maleimido group can be introduced into an end of the polyalkylene glycol condensed with an appropriate phospholipid such as stearylated polyethylene glycol, and a reactive functional group such as thiol group, amino group or hydroxyl group of the target cell-selective ligand can be reacted with the maleimido group. When an oligopeptide is used as the target cell-selective ligand, it is preferable, for example, to react thiol group of a cysteine (Cys) residue at the N- or C-terminus of the oligopeptide. Typical examples of the reaction are shown in the examples mentioned in the specification. For example, a liposome modified with PEG bound with transferrin is disclosed in J. Pharm., 281, pp. 25-33, 2004, and a liposome modified with PEG bound with Fab-fragmented antibody is disclosed in J. Pharm., 342, 194-200, 2007. Therefore, those skilled in the art can easily bind a ligand to PEG and modify a lipid membrane structure with the ligand bound to PEG by referring to these publications. Amount of the target cell-selective ligand to be bound is not particularly limited, and can be appropriately chosen depending on types of the ligand and the receptor, binging force, binding specificity of the ligand and the receptor, type of the target cell, type of the substance to be delivered, and the like. For example, an appropriate modification amount can be determined for an arbitrary ligand by the specific method described in the examples mentioned in the specification. For example, modification with a polyalkylene glycol bound with a target cell-selective ligand in an amount of about 10 to 15 mol % may provide a preferred result.

Surface of the lipid membrane structure of the present invention is modified with a polypeptide containing plural contiguous arginine residues (polyarginine). As the polyarginine, preferably a polypeptide containing 4 to 20 contiguous arginine residues, more preferably a polypeptide consisting only of 4 to 20 contiguous arginine residues, most preferably octaarginine, and the like can be used. It is known that, by modifying surface of a lipid membrane structure such as liposome with a polyarginine such as octaarginine, intracellular delivery efficiency of a target substance encapsulated in liposome can be improved (Journal of Controlled Release, 98, pp. 317-323, 2004; International Patent Publication WO2005/32593). Surface of the lipid membrane structure can be easily modified with a polyarginine according to the method described in the aforementioned publications using, for example, a lipid-modified polyarginine such as stearylated octaarginine as a constituent lipid of the lipid membrane structure. The disclosures of the aforementioned publications and the disclosures of all of the references cited in the specification are incorporated into the disclosure of this specification by reference. Although amount of the polyarginine used for the surface modification can be appropriately determined by referring to the aforementioned publications, it is preferable to appropriately choose the amount so as to be within such a range that the retainability in blood is not substantially affected, and amount of the lipid membrane structure to be taken up into cells is maximized. For example, the amount can be chosen so as to be 15 mol % or less, and an amount of about 5 mol % may be more preferred. Further, by binding a polyarginine to a polyethylene glycol, surface modification with polyarginine and surface modification with polyethylene glycol can also be simultaneously attained.

In order to promote permeation of the lipid membrane structure of the present invention into nuclei, surface of the lipid membrane structure can also be modified with, for example, a tri- or higher oligosaccharide compound. Although type of the tri- or higher oligosaccharide compound is not particularly limited, for example, an oligosaccharide compound comprising about 3 to 10 of linked saccharide units can be used, and an oligosaccharide compound comprising about 3 to 6 of linked saccharide units can be preferably used.

More specifically, examples of the oligosaccharide compound include, for example, trisaccharide compounds such as cellotriose (β-D-glucopyranosyl-(1->4)-β-D-glucopyranosyl-(1->4)-D-glucose), chacotriose (α-L-rhamnopyranosyl-(1->2)-[α-L-rhamnopyranosyl-(1->4)]-D-glucose), gentianose (β-D-fructofuranosyl-β-D-glucopyranosyl-(1->6)-α-D-glucopyranoside), isomaltotriose (α-D-glucopyranosyl-(1->6)-α-D-glucopyranosyl-(1->6)-D-glucose), isopanose (α-D-glucopyranosyl-(1->4)-[α-D-glucopyranosyl-(1->6)]-D-glucose), maltotriose (α-D-glucopyranosyl-(1->4)-α-D-glucopyranosyl-(1->4)-D-glucose), manninotriose (α-D-galactopyranosyl-(1->6)-α-D-galactopyranosyl-(1->6)-D-glucose), melezitose (α-D-glucopyranosyl-(1->3)-8-D-fructofuranosyl=α-D-glucopyranoside), panose (α-D-glucopyranosyl-(1->6)-α-D-glucopyranosyl-(1->4)-D-glucose), planteose (α-D-galactopyranosyl-(1->6)-β-D-fructofuranosyl=α-D-glucopyranoside), raffinose (8-D-fructofuranosyl=α-D-galactopyranosyl-(1->6)-α-D-glucopyranoside), solatriose (α-L-rhamnopyranosyl-(1->2)-[8-D-glucopyranosyl-(1->3)]-D-galactose), and umbelliferose (β-D-fructofuranosyl=α-D-galactopyranosyl-(1->2)-α-D-galactopyranoside; tetrasaccharide compounds such as lycotetraose (β-D-glucopyranosyl-(1->2)-[β-D-xylopyranosyl-(1->3)]-β-D-glucopyranosyl-(1->4)-8-D-galactose, maltotetraose (α-D-glucopyranosyl-(1->4)-α-D-glucopyranosyl-(1->4)-α-D-glucopyranosyl-(1->4)-D-glucose), and stachyose (β-D-fructofuranosyl=α-D-galactopyranosyl-(1->6)-α-D-galactopyranosyl-(1->6)-α-D-glucopyranoside); pentasaccharide compounds such as maltopentaose (α-D-glucopyranosyl-(1->4)-α-D-glucopyranosyl-(1->4)-α-D-glucopyranosyl-(1->4)-α-D-glucopyranosyl-(1->4)-D-glucose, and verbascose (β-D-fructofuranosyl-α-D-galactopyranosyl-(1->6)-α-D-galactopyranosyl-(1->6)-α-D-galactopyranosyl-(1->6)-α-D-glucopyranoside; and hexasaccharide compounds such as maltohexaose (α-D-glucopyranosyl-(1->4)-α-D-glucopyranosyl-(1->4)-α-D-glucopyranosyl-(1->4)-α-D-glucopyranosyl-(1->4)-α-D-glucopyranosyl (1->4)-D-glucose), but the oligosaccharide compound is not limited to these examples.

Oligosaccharide compounds as trimer to hexamer of glucose can be preferably used, and oligosaccharide compounds as trimer or tetramer of glucose can be more preferably used. More specifically, isomalttriose, isopanose, maltotriose, maltotetraose, maltopentaose, maltohexaose, and the like can be preferably used, and among these, maltotriose, maltotetraose, maltopentaose, and maltohexaose consisting of glucose units linked through α1-4 linkages are more preferred. Particularly preferred are maltotriose and maltotetraose, and most preferred is maltotriose. Although amount of the oligosaccharide compound used for the surface modification of the lipid membrane structure is not particularly limited, the amount is, for example, about 1 to 30 mol %, preferably about 2 to 20 mol %, more preferably about 5 to 10 mol %, based on the total amount of lipids.

Although the method for modifying the surface of the lipid membrane structure with the oligosaccharide compound is not particularly limited, for example, since liposomes consisting of lipid membrane structures of which surfaces are modified with monosaccharides such as galactose and mannose are known (International Patent Publication WO2007/102481), the surface modification method described in this publication can be employed. The entire disclosure of the aforementioned publication is incorporated into the disclosure of this specification by reference. This means is a method of binding a monosaccharide compound to polyalkylene glycolated lipids to perform surface modification of lipid membrane structures. Since surfaces of lipid membrane structures can be simultaneously modified with polyalkylene glycol by this means, this method is preferred.

As lipid derivatives for enhancing retainability in blood used for the preparation of the lipid membrane structure of the present invention, for example, glycophorin, ganglioside GM1, phosphatidylinositol, ganglioside GM3, glucuronic acid derivative, glutamic acid derivative, polyglycerin-phospholipid derivative, and the like can be used. As hydrophilic polymer for enhancing retainability in blood, besides polyalkylene glycol, dextran, pullulan, Ficoll, polyvinyl alcohol, styrene-maleic anhydride alternating copolymer, divinyl ether-maleic anhydride alternating copolymer, amylose, amylopectin, chitosan, mannan, cyclodextrin, pectin, carrageenan, and the like can also be used for the surface modification.

In order to efficiently extricate the lipid membrane structure from the inside of the endosome into the cytoplasm, the lipid membrane of the lipid membrane structure of the present invention may be modified with GAL4. For example, since liposomes of which surfaces are modified with GAL4 is described in Japanese Patent Unexamined Publication (KOKAI) No. 2006-28030, the lipid membrane structure of which surface is modified with GAL4 can be easily prepared according to the method described in the aforementioned publication. In general, by preparing a lipid membrane structure using a cholesterol derivative of GAL4 (Chol-GAL4) as a lipid component, a lipid membrane structure of which surface is modified with GAL4 can be prepared. Although amount of GAL4 used for the surface modification of the lipid membrane structure is not particularly limited, the amount is, for example, about 0.01 to 10 mol %, preferably about 0.1 to 4 mol %, more preferably about 1 to 3 mol %, based on the total amount of lipids.

The term “GAL4” referred to in the specification include the peptide specified by SEQ ID NO: 1 mentioned in Sequence Listing of Japanese Patent Unexamined Publication (KOKAI) No. 2006-28030, as well as a modified peptide thereof having the amino acid sequence of the aforementioned peptide, but including deletion, substitution and/or addition of one or several amino acid residues, and having substantially the same properties as those of GAL4 (for example, a property of capable of fusing with lipid membranes under an acid condition). The term “GAL4” used in the specification should not be construed in any limitative way. As for GAL4 and method for surface modification of a lipid membrane structure with GAL4, the entire disclosure of Japanese Patent Unexamined Publication (KOKAI) No. 2006-28030 is incorporated into the disclosure of this specification by reference.

Surface of the lipid membrane structure of the present invention may also be modified with an MPC polymer. The MPC polymer is that obtainable by polymerizing 2-methacryloyloxyethylphosphorylcholine (MPC). It has been demonstrated that, since this polymer has a molecular structure similar to that of biomembranes, it scarcely shows interactions with biological substances such as polypeptides and hemocytes, and shows superior biocompatibility. In the specification, the term “MPC polymer” is used for referring to both a homopolymer of MPC and a copolymer of MPC and another polymerization component.

As the MPC polymer, commercial polymers can be easily obtained. For example, a homopolymer of MPC (CAS: 67881-99-6); a copolymer of MPC with butyl methacrylate (CAS: 125275-25-4); a terpolymer of MPC, sodium methacrylate and butyl methacrylate; a bipolymer of MPC and 2-hydroxy-3-(meth)acryloyloxypropyl-trimethylammonium chloride; a phospholipid polymer (LIPIDURE-S), and the like are provided by NOF Corporation with the registered trademark of “LIPIDURE”, and any of these can be used for the present invention.

Although type of the MPC polymer used for the present invention is not particularly limited, for example, a copolymer of MPC and a methacrylic acid ester such as butyl methacrylate, especially such a block copolymer, and the like can be preferably used. The preparation method of this copolymer is described in detail in Japanese Patent No. 2890316, and those skilled in the art can easily prepare a desired copolymer by referring to this patent publication. The entire disclosure of this patent publication is incorporated into the disclosure of this specification by reference. In the present invention, an MPC polymer having water solubility and also having a hydrophobic group is preferably used. From this point of view, an MPC copolymer prepared by using an acrylic acid ester or methacrylic acid ester having about 4 to 18 carbon atoms can be preferably used. As a copolymer of MPC and butyl methacrylate (BMA), for example, a copolymer having a molar ratio of MPC and BMA of 5:5 (PMB50), a copolymer having a molar ratio of MPC and BMA of 3:7 (PMB30), and the like are known, and it can be easily prepared according to, for example, the method described in Polymer Journal, 22, pp. 355-360, 1990, or the like (specific preparation methods are explained in, for example, Japanese Patent Unexamined Publication (KOKAI) No. 2007-314526). For the present invention, PMB50 can be particularly preferably used. Although degree of polymerization and molecular weight of the MPC polymer are not particularly limited, a polymer having an average molecular weight (weight average molecular weight) of, for example, about 5,000 to 300,000, preferably about 10,000 to 100,000, can be used from a viewpoint of maintaining water solubility.

Although the method for modifying the lipid membrane structure with the MPC polymer is not particularly limited, for example, an MPC polymer can be added to an aqueous dispersion of the lipid membrane structure such as liposomes, and the mixture can be left at room temperature for about several minutes to several hours. Although amount of the MPC polymer to be added to the aforementioned aqueous dispersion is not particularly limited, the polymer may be added in an amount corresponding to the amount of the MPC polymer used for the modification, for example, 0.01 to 1 mass %, preferably about 0.1 to 10 mass %, more preferably about 0.1 to 3 mass %, based on the total amount of lipids of the lipid membrane structure. By this operation, the MPC polymer is quickly taken up into the lipid components of the lipid membrane structure, and the lipid membrane structure of which surface is modified with the MPC polymer can be prepared. Although amount of the MPC polymer used for the surface modification is not particularly limited, the amount is, for example, in the range of about 0.1 to 5 mass % based on the total amount of lipids of the lipid membrane structure.

The lipid membrane structure of the present invention may contain one or two or more kinds of substances selected from the group consisting of a membrane stabilization agent such as sterol, glycerol, and a fatty acid ester thereof, an antioxidant such as tocopherol, propyl gallate, ascorbyl palmitate, and butylated hydroxytoluene, a chargeable substance, a membrane polypeptide, and the like. Examples of the chargeable substance that imparts positive charge include saturated or unsaturated fatty amines such as stearylamine and oleylamine; saturated or unsaturated synthetic cationic material such as dioleoyltrimethylammonium propane; cationic polymers, and the like, and examples of the chargeable substance that imparts negative charge include, for example, dicetyl phosphate, cholesteryl hemisuccinate, phosphatidylserine, phosphatidylinositol, phosphatidic acid, and the like. Examples of the membrane polypeptide include, for example, extrinsic membrane polypeptides and integral membrane polypeptides. Amounts of these substances to be added are not particularly limited, and can be appropriately chosen depending on the purpose.

Further, the lipid membrane structure of the present invention may be imparted with one or two or more functions selected from, for example, temperature change-sensing function, membrane permeating function, gene expressing function, pH sensing function, and the like. By appropriately imparting these functions, retainability in blood of the lipid membrane structure encapsulating, for example, a nucleic acid containing a gene or the like can be improved, a rate of capture by reticuloendothelial systems of liver, spleen and the like can be reduced, the lipid membrane structure can be efficiently extricated from the endosome and transferred to the cytoplasm after endocytosis of a target cell, and further, it also becomes possible to attain high gene expression activity in a nucleus.

Examples of temperature change-sensitive lipid derivatives that can impart the temperature change-sensing function include, for example, dipalmitoylphosphatidylcholine and the like. Examples of pH-sensitive lipid derivatives that can impart the pH sensing function include, for example, dioleoylphosphatidylethanolamine and the like.

Surface of the lipid membrane structure of the present invention may be further modified with a ligand that can specifically bind with a receptor on surface of a target cell, as required. For example, a monoclonal antibody directed to a biological component specifically expressed in a target cell, tissue, or organ or the like can be disposed on the surface of the lipid membrane structure as a ligand. This technique is described in, for example, STEALTH LIPOSOME (pages 233 to 244, published by CRC Press, Inc., edited by Danilo Lasic and Frank Martin) and the like As a component of the lipid membrane structure, there can be contained a lipid derivative that can react with mercapto group in a monoclonal antibody or a fragment thereof (e.g., Fab fragment, F(ab′)₂ fragment, Fab′ fragment and the like), specifically, a lipid derivative having a maleinimide structure such as poly(ethylene glycol)-α-distearoylphosphatidylethanolamine-ω-maleinimide and α-[N-(1,2-distearoyl-sn-glycero-3-phosphorylethyl)carbamyl]-w-{3-[2-(2,5-dihydro-2,5-dioxo-1H-pyrrol-1-yl)ethanecarboxamido]propyl}-poly(oxy-1,2-ethanediyl), and thereby the monoclonal antibody can be bound to the surface of the membrane of the lipid membrane structure.

Furthermore, surface of the lipid membrane structure of the present invention may be modified with INF7. INF7 is a glutamic acid-rich peptide obtained by modifying a peptide (1-23) derived from the influenza HApolypeptide (HA2), and it has been reported that the lipid structure of liposomes is collapsed in the presence of INF7, and a substance encapsulated in the liposomes is easily released (Biochemistry, 46, pp. 13490-13504, 2007). There has also been proposed a delivery system comprising polyethylene glycol tetraacrylate (PEG-TA) bound with INF7 (The Journal of Gene Medicine, 10, pp. 1134-1149, 2008). Those skilled in the art can easily use INF7 in the present invention by referring to these publications. The term “INF7” used in the specification is used to refer to the peptide specified by the sequence described in Biochemistry, 46, pp. 13490-13504, 2007, Table 1, as well as a modified peptide thereof having the amino acid sequence of the aforementioned peptide, but including deletion, substitution and/or addition of one or several amino acid residues, and having substantially the same properties as those of INF7. The term “INF7” used in the specification should not be construed in any limitative way. The disclosures of the aforementioned publications and the disclosures of all of the references cited in the specification are incorporated in the disclosure of this specification by reference.

Although the method for modifying the lipid membrane structure with INF7 is not particularly limited, the lipid membrane structure of which surface is modified with INF7 can generally be easily prepared by constructing the lipid membrane structure by using a lipid-modified INF7 comprising a lipid compound and INF7 covalently bound with each other as a lipid membrane constituent lipid. As the lipid-modified INF, for example, stearylated INF7 and the like can be used. This compound can be easily prepared according to the method described in Futaki, S. et al., Bioconjug. Chem., 12 (6), pp. 1005-1011, 2001. Although amount of INF7 used for the surface modification is not particularly limited, the amount is generally in the range of 1 to 5 mole %, preferably about 3 to 5 mole %, based on the total amount of lipids of the lipid membrane structure.

A multifunctional envelope-type nano device (MEND) is known, and it can be preferably used as the lipid membrane structure of the present invention. MEND has, for example, a structure that it contains a complex of a nucleic acid such as plasmid DNA and a cationic polymer such as protamine as a core, and the core is encapsulated in the inside of a lipid envelope membrane in the form of liposome. On the lipid envelope membrane of MEND, a peptide for adjusting pH responding property and membrane permeability can be disposed as required, and the external surface of the lipid envelope membrane can be modified with an alkylene glycol such as polyethylene glycol. Condensed DNA and the cationic polymer are encapsulated in the inside of the lipid envelope of MEND, and it is designed so that gene expression can be efficiently attained. As MEND suitably used for the present invention, MEND in which a complex of a plasmid DNA incorporated with a desired gene and protamine is encapsulated in the inside, and the outer surface of the lipid envelope is modified with an oligosaccharide-bound PEG is preferred. For the modification with the oligosaccharide-bound PEG, it is preferable to use stearylated polyethylene glycol bound with the polypeptide (a) and/or the polypeptide (b) mentioned above as a constituent lipid component. As for MEND, for example, references for general remarks, such as Drug Delivery System, 22-2, pp. 115-122, 2007, can be referred to. The disclosure of the aforementioned publication and the disclosures of all of the references cited in the specification are incorporated into the disclosure of this specification by reference.

Although form of the lipid membrane structure is not particularly limited, examples include, for example, a dispersion in an aqueous solvent (for example, water, physiological saline, phosphate buffered physiological saline, and the like), a lyophilized product of the aqueous dispersion, and the like.

The method for preparing the lipid membrane structure is not particularly limited, either, and an arbitrary method available for those skilled in the art can be employed. For example, the lipid membrane structure can be prepared by dissolving all the lipid components in an organic solvent such as chloroform, forming a lipid membrane by exsiccation under reduced pressure in an evaporator or spray drying using a spray dryer, then adding an aqueous solvent to the aforementioned dried mixture, and emulsifying the mixture with an emulsifier such as homogenizer, an ultrasonic emulsifier, a high pressure injection emulsifier, or the like. Further, it can be prepared by a method well known as a method for preparing liposomes, for example, the reverse phase evaporation method, and the like. When it is desired to control the size of the lipid membrane structure, extrusion (extrusion filtration) can be performed under high pressure by using a membrane filter having pores of uniform diameters. Although size of the dispersed lipid membrane structure is not particularly limited, in the case of liposome, for example, particle size is about 50 nm to 5 μm, preferably about 50 nm to 400 nm, more preferably 50 nm to 300 nm. In the case of liposome encapsulating a medicament such as antitumor agent, it may be preferred that the particle size is about 200 nm to 400 nm, and it may be particularly preferred that the particle size is about 300 nm. Further, it may be further preferred that the particle size is about 150 nm to 250 nm. The particle size can be measured by, for example, the DLS (dynamic light scattering) method.

The composition of the aqueous solvent (dispersion medium) is not particularly limited, and examples include, for example, a buffer such as phosphate buffer, citrate buffer, and phosphate-buffered physiological saline, physiological saline, a medium for cell culture and the like. Although the lipid membrane structure can be stably dispersed in these aqueous solvents (dispersion media), the solvents may be further added with a saccharide (aqueous solution), for example, a monosaccharide such as glucose, galactose, mannose, fructose, inositol, ribose and xylose, a disaccharide such as lactose, sucrose, cellobiose, trehalose and maltose, a trisaccharide such as raffinose and melezitose, and polysaccharide such as cyclodextrin, a sugar alcohol such as erythritol, xylitol, sorbitol, mannitol, and maltitol, or a polyhydric alcohol (aqueous solution) such as glycerin, diglycerin, polyglycerin, propylene glycol, polypropylene glycol, ethylene glycol, diethylene glycol, triethylene glycol, polyethylene glycol, ethylene glycol mono-alkyl ether, diethylene glycol mono-alkyl ether and 1,3-butylene grycol. In order to stably store the lipid membrane structure dispersed in such an aqueous solvent for a long period of time, it is desirable to minimize electrolytes in the aqueous solvent from a viewpoint of physical stability such as prevention of aggregation. Further, from a viewpoint of chemical stability of lipids, it is desirable to control pH of the aqueous solvent to be in a range of from weakly acidic pH to around neutral pH (pH 3.0 to 8.0), and/or to remove dissolved oxygen by nitrogen bubbling or the like.

When the resulting aqueous dispersion of the lipid membrane structure is lyophilized or spray-dried, use of a saccharide (aqueous solution), for example, a monosaccharide such as glucose, galactose, mannose, fructose, inositol, ribose and xylose, a disaccharide such as lactose, sucrose, cellobiose, trehalose and maltose, a trisaccharide such as raffinose and melezitose, a polysaccharide such as cyclodextrin, a sugar alcohol such as erythritol, xylitol, sorbitol, mannitol, and maltitol or the like may improve stability. When the aforementioned aqueous dispersion is frozen, use of the aforementioned saccharide or a polyhydric alcohol (aqueous solution) such as glycerin, diglycerin, polyglycerin, propylene glycol, polypropylene glycol, ethylene glycol, diethylene glycol, triethylene glycol, polyethylene glycol, ethylene glycol mono-alkyl ether, diethylene glycol mono-alkyl ether and 1,3-butylene glycol may improve stability.

In the inside of the lipid membrane structure of the present invention, for example, liposome, a substance to be delivered to inside of a target cell, preferably inside of a nucleus of a target cell, can be encapsulated. Although type of the substance to be encapsulated is not particularly limited, active ingredients of arbitrary medicaments such as antitumor agent, anti-inflammatory agent, antimicrobial agent, and antiviral agent as well as other arbitrary substances such as saccharides, peptides, nucleic acids, low molecular weight compounds, and metallic compounds can be encapsulated. As the antitumor agent, antitumor agents already clinically used, for example, mesotrexate, doxorubicin, cisplatin, and the like, can be preferably used. A liposome preparation encapsulating doxorubicin as an antitumor agent is already put into practical use, and widely used on clinical sites as an injection (“Doxil” (registered trademark), Janssen Pharmaceutical), and therefore a medicament consisting of the lipid membrane structure of the present invention encapsulating doxorubicin can be used by, for example, an administration method and at a dose similar to those for Doxil. Examples of the nucleic acid include a nucleic acid containing a gene, and specific examples include, for example, a gene incorporated into a plasmid. However, the nucleic acid is not limited to these specific examples. Further, it should be understood that arbitrary genes can be used as the gene. Although a case of enclosing a nucleic acid will be specifically explained below as an example of the present invention, the scope of the present invention is not limited to this specific embodiment.

In the lipid membrane structure of the present invention, a nucleic acid can be preferably encapsulated. The nucleic acid includes DNA and RNA, as well as analogues and derivatives thereof (for example, peptide nucleic acid (PNA), phosphorothioate DNA, and the like). The nucleic acid may be a single-stranded or double-stranded nucleic acid, and may be a linear or cyclic nucleic acid. The nucleic acid may contain a gene. The gene may be any of oligonucleotide, DNA, and RNA, and in particular, genes for in vitro induction such as transformation, genes acting after in vivo expression thereof, for example, genes for gene therapies such as normal genes for homologous recombination, and the like can be mentioned. As the nucleic acid for therapeutic treatment, antisense oligonucleotide, antisense DNA, antisense RNA, and a gene coding for an enzyme or a physiologically active substance such as cytokine, as well as a nucleic acid having a function of controlling gene expression, for example, a functional nucleic acid including RNA such as siRNA can also be used, and these are also encompassed within the scope of the term “nucleic acid” used in the specification. The term “nucleic acid” used in the specification must be construed in its broadest sense, and the term should not be construed in any limitative way.

Further, when a nucleic acid is encapsulated in the lipid membrane structure of the present invention, a compound having a nucleic acid-introducing function can also be added. Examples of such a compound include, for example, O,O′-N-didodecanoly-N-(α-trimethylammonioacetyl)-diethanolamine chloride, O,O′-N-ditetradecanoly-N-(α-trimethylammonioacetyl)-diethanolamine chloride, O,O′-N-dihexadecanoly-N-(α-trimethylammonioacetyl)-diethanolamine chloride, O,O′-N-dioctadecenoyl-N-(α-trimethylammonioacetyl)-diethanolamine chloride, O,O′,O″-tridecanoly-N-(ω-trimethylammoniodecanoyl)aminomethane bromide, N-[α-trimethylammonioacetyl]-didodecyl-D-glutamate, dimethyldioctadecylammonium bromide, 2,3-dioleyloxy-N-[2-(sperminecarboxamido)ethyl]-N,N-dimethyl-1-propane ammonium trifluoroacetate, 1,2-dimyristyloxypropyl-3-dimethyl-hydroxyethylammonium bromide, 3-8-[N—(N′,N′-dimethylaminoethane)-carbamoyl]cholesterol, and the like. These compounds having a nucleic acid-introducing function may be disposed at an arbitrary position of the membrane of the lipid membrane structure, and/or filled in the inside of the lipid membrane structure.

For example, the lipid membrane structure encapsulating a nucleic acid can be used as a carrier for delivering the nucleic acid into a nucleus of a target cell. When aiming at gene expression, it is particularly preferable to use a nucleic acid containing a desired gene as the nucleic acid and the aforementioned MEND. For example, by administering a lipid membrane structure, preferably MEND, enclosing a nucleic acid containing a gene to a mammal including human, a desired gene can be delivered into nuclei of desired target cells, and efficiently expressed. Although administration method is not particularly limited, parenteral administration is preferred, and intravenous administration is more preferred. When the lipid membrane structure of the present invention is used as a medicament, for example, a medicament in the form of a pharmaceutical composition can be prepared with appropriate pharmaceutical additives and administered.

Hereafter, the present invention will be more specifically explained with reference to examples. However, the scope of the present invention is not limited to the following examples.

### Example 1

### (1) Materials and Methods

### (a) Preparation of Liposomes Using Peptide Ligand

A ligand peptide having a cysteine residue including thiol group at the end (CYGGRGNG) and a PEG-lipid derivative having maleimido group at the end, Mal-PEG-DSPE, were mixed at a ratio of 1:1 (molar ratio), and the mixture was shaken for 24 hours to obtain a peptide-bound PEG-lipid derivative, Pep-PEG-DSPE. Liposomes were prepared with three kinds of lipids, egg yolk phosphatidylcholine (henceforth abbreviated as “EPC”), cholesterol (henceforth abbreviated as “Chol”), and rhodamine-labeled 1,2-dioleyl-sn-glycero-3-phosphoethanolamine (henceforth abbreviated as “Rho-DOPE”), and added with necessary amounts of Pep-PEG-DSPE and stearylated tetrarginine (henceforth abbreviated as “STR-R4”) according to the post-modification method to prepare liposomes.

First, lipid solutions (ethanol solutions of EPC and Chol, and chloroform solution of Rho-DOPE) were put into a glass test tube in a total amount of 600 nmol/600 μL, and added with an equal volume of chloroform, and the solvent was evaporated under a nitrogen gas atmosphere or under reduced pressure. The resulting lipid films were added with PBS so that the lipid concentration became 0.4 mM, hydrated at room temperature for 10 minutes, then ultrasonicated on a bath type sonicator, and further ultrasonicated with a probe type sonicator to prepare SUV liposomes.

The liposome solution was added with a necessary volume of the STR-R4 aqueous solution, the mixture was shaken at 55° C. for 30 minutes to modify the liposomes with STR-R4, then added with a necessary amount of PEG-DSPE or Pep-PEG-DSPE, and the mixture was further shaken at 55° C. for 30 minutes to attain modification with PEG. The particle size and zeta potential were measured by the DLS (dynamic light scattering) method.

### (b) Preparation of Liposomes Using Protein Ligand

Core particles consisting of plasmid DNA (pDNA) condensed with polyethyleneimine (PEI) were encapsulated in lipid envelopes containing three kinds of lipids, 1,2-dioleyl-sn-glycero-3-phosphoethanolamine (henceforth abbreviated as “DOPE”), Chol and stearylated octaarginine (henceforth abbreviated as “STR-R8”) to prepare pDNA-encapsulated liposomes. The base lipid composition was adjusted to a composition ratio of DOPE:Chol:STR-R8=70:20:10 (molar ratio).

The pDNA core particles were prepared by mixing pDNA containing a firefly luciferase gene (7,037 bp) and PEI at a+/−ratio of 0.8. Solutions of pDNA and PEI in mM HEPES (pH 7.4) were prepared, and then 200 μl of the plasmid DNA solution (0.075 mg/ml) put into a vortex mixer was slowly added dropwise with 100 μl of the PEI solution (2.4 mM) to gradually mix them. The mixture was further left standing at room temperature for 15 minutes to prepare the core particles.

The chloroform and ethanol solutions (3:1 (v/v)) containing 90 nmol of the lipids were added to a glass test tube, and the solvents were evaporated under reduced pressure to obtain the lipid films. The lipid films were added with 300 μl of the core particle solution, and the mixture was left standing at room temperature for 15 minutes to hydrate the films, and ultrasonicated for about 1 minute to obtain pDNA core-encapsulated R8-modified liposomes. Further, the pDNA core-encapsulated liposome solution was added with a 10 mM HEPES (pH 7.4) solution containing necessary amount of PEG-DSPE or Mal-PEG-DSPE, and the mixture was left standing at room temperature for 30 minutes to perform modification with PEG.

Transferrin (Tf) (125 mM) and SPDP (132 mM) were dissolved in PBS, and the solution was shaken at room temperature for 30 minutes. Then, a PDP-bound Tf fraction was collected by gel filtration using Sephadex G-25. The PDP-bound Tf fraction was added with DTT (100 mM), and the mixture was shaken at room temperature for 30 minutes to perform the reduction reaction. The reducing agent was removed by gel filtration using Sephadex G-25 to collect a reduced type Tf (SH-Tf) fraction, and collected amount was measured by quantifying proteins. The pDNA core-encapsulated liposomes modified with Mal-PEG-DSPE were added with SH-Tf (at a ratio of 16 μg of SH-Tf to 1 μg of pDNA), and the mixture was shaken overnight at 4° C. The reaction mixture was subjected to ultracentrifugation at 4° C. and 30000×g for 2 hours to remove unreacted Tf in the supernatant, and the precipitated Tf-modified pDNA-encapsulated liposome fraction was collected. The liposomes were suspended in a 10 mM HEPES (pH 7.4) solution, and the nucleic acid was quantified by staining with SDOC (5 mM) and PI (100 μg/ml). The particle size and zeta potential were measured by the DLS method.

### (c) Evaluation of Cellular Uptake

MS-1 cells were inoculated on a 24-well plate at a cell density of 4×10⁴ cells/well, wells were washed with PBS(−) 24 hours afterward, and 500 μl/well of Krebs buffer containing the fluorescence-labeled liposomes (lipid concentration: 0.12 mM) was added, and incubation was performed at 37° C. for 3 hours. The liposomes were removed, the cells were washed with Krebs buffer, then Reporter Lysis Buffer was added (701/well), and the cells were left standing at −80° C. for 20 minutes or more. After thawing, the cells were collected with a cell scraper, and centrifuged at 4° C. and 15000 rpm for 5 minutes. The supernatant in a volume of 50 μL was diluted twice with purified water, and then fluorescence intensity was measured (Ex./Em.=555/575 nm).

### (d) Evaluation of Gene Expression

HeLa cells were inoculated on a 24 well-plate at a cell density of 4×10⁴ cells/well, the wells were washed with PBS(−) 24 hours afterward, 3001/well (0.4 μg pDNA/well) of DMEM containing 10% serum added with the pDNA-encapsulated liposomes was added, and then incubation was performed at 37° C. for 3 hours. The DMEM containing the pDNA-encapsulated liposomes was removed, 1 ml/well of DMEM containing 10% serum was added, and incubation was performed at 37° C. for 21 hours. After washing with PBS, Reporter Lysis Buffer (70 μl/well) was added, and the cells were left standing at −80° C. for 20 minutes or more. After thawing, the cells were collected with a cell scraper, and centrifuged at 4° C. and 15000 rpm for 5 minutes, and then expression amount of the luciferase was calculated by measuring the luciferase activity and the protein amount in the supernatant.

### (2) Results

### (a) Cellular Uptake of Liposomes by Peptide Ligand

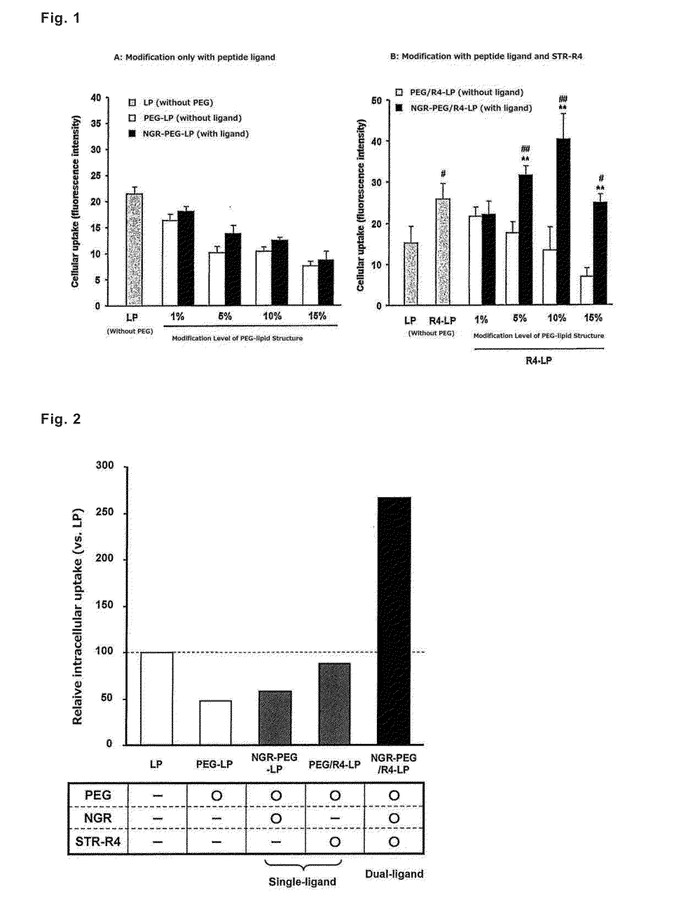

The prepared liposomes had a particle size of about 100 nm irrespective of the composition thereof, and showed a substantially neutral zeta potential (Table 1). It is known that a peptide having the NGR motif used as the peptide ligand specifically recognizes CD13, which is a tumor vascular endothelial marker. The cellular uptake amount of the liposomes modified with the NGR motif-PEG lipid derivative having the NGR motif as the ligand did not significantly differ from that observed with modification with the PEG lipid not having the ligand (FIG. 1A, white bars vs. black bars). Further, the uptake amount decreased in a PEG modification amount-dependent manner (FIG. 1A). Further, the uptake amount increased when the liposomes were modified with 0.25 mol % of STR-R4, but when the liposomes were modified with PEG, the uptake amount decreased in a concentration-dependent manner (FIG. 1B, white bars). On the other hand, when the NGR ligand was bound to PEG and used to modify the liposomes together with STR-R4, the amount of cellular uptake increased up to 10 mol % (FIG. 1B, black bars).

TABLE 1

| | PEG-LP | | NGR-PEG-LP | | PEG/R4-LP | | NGR-PEG/R4-LP | |
|---|---|---|---|---|---|---|---|---|
| | | zeta- | | zeta- | | zeta- | | zeta- |
| | | potential | | potential | | potential | | potential |
| Conc. of PEG-lipid | Size (nm) | (mV) | Size (nm) | (mV) | Size (nm) | (mV) | Size (nm) | (mV) |
| 0% | 97 ± 5 | 0.2 ± 0.1 | — | — | 89 ± 5 | 2.2 ± 1 | — | — |
| 1% | 101 ± 2 | 0.2 ± 0.2 | 103 ± 1 | −1.6 ± 0.9 | 91 ± 8 | 0.8 ± 0.5 | 91 ± 8 | 1.3 ± 0.8 |
| 2% | 100 ± 6 | −5.0 ± 2.4 | 103 ± 8 | 1.5 ± 0.7 | 93 ± 2 | −0.1 ± 0.1 | 99 ± 3 | 0.8 ± 0.6 |
| 5% | 103 ± 8 | −0.8 ± 0.1 | 102 ± 2 | −0.8 ± 0.7 | 92 ± 3 | −0.2 ± 0.1 | 91 ± 2 | −0.1 ± 0.1 |
| 10% | 100 ± 5 | −1.5 ± 0.4 | 102 ± 6 | −2.5 ± 0.8 | 90 ± 4 | −1.6 ± 0.3 | 88 ± 3 | −0.9 ± 0.1 |
| 15% | 100 ± 4 | −2.1 ± 0.5 | 99 ± 2 | −3.6 ± 0.6 | 85 ± 7 | −3.2 ± 0.6 | 93 ± 6 | −1.2 ± 0.8 |

(b) Cellular Uptake Amounts of the Liposomes at a PEG Concentration of 10 Mol %, which was the optimal modification condition in the case of CD13-expressing model MS-1 cells, are shown in FIG. 2 in terms of relative values based on the value obtained for the PEG-unmodified liposomes. The amount of cellular uptake decreased by PEG modification was slightly increased by modification with either NGR or STR-R4, but the effect was not significant, and the amount did not exceed that obtained without modification with PEG. Whist, it was demonstrated that when the NGR ligand was bound to PEG and used to modify the liposomes together with STR-R4, the amount of cellular uptake significantly increased. This result indicates that the target cell-selective ligand and tetrarginine as the cell membrane permeable peptide (CPP) synergistically function without being affected by the modification with PEG, and it is clearly understood that the liposome of the present invention can exhibit specificity for a target cell and function of delivering a medicament into cells without being influenced by modification with PEG.

### (c) Gene Expression Activity of Liposomes Using Protein Ligand

The mean particle diameter of the pDNA core was 88±6 nm, and the zeta potential thereof was −25±8 mV. Although the pDNA core-encapsulated R8 liposomes had high zeta potential, the zeta potential decreased in a PEG modification amount-dependent manner (Table 2). Further, although the particle size slightly decreased by the modification with PEG, significant change was not observed. The gene expression activity obtained by transfecting these pDNA core-encapsulated R8 liposomes into the HeLa cells is shown in FIG. 3. By the modification with PEG, the gene expression activity of the R8 liposomes was significantly decreased.

TABLE 2

| | PEG Concentration (mol %) | | | | |
|---|---|---|---|---|---|
| | 0 | 5 | 10 | 15 | 20 |
| Particle | 261 ± 21 | 208 ± 21 | 216 ± 41 | 218 ± 16 | 224 ± 29 |
| Diameter | | | | | |
| (nm) | | | | | |
| zeta- | 41 ± 3 | 21 ± 4 | 9 ± 3 | 4 ± 1 | −3 ± 5 |
| potential | | | | | |
| (mV) | | | | | |

It is known that Tf is overexpressed in cancer cells. Tf-modified R8 liposomes were prepared by binding Tf as a target cell-selective ligand to the tip end portion of PEG of pDNA-encapsulated R8 liposomes. The particle size and zeta potential of these liposomes are shown in Table 3. Although the particle size was not changed by the modification with Tf, the zeta potential shifted from the neutral range to the negative range. This result was observed because Tf was negatively charged, and indicated that the liposomes were modified with Tf bound to PEG.

TABLE 3

| | PEG Concentration (mol %) | | | | | |
|---|---|---|---|---|---|---|
| | 10 | | 15 | | 20 | |
| | Tf Modification | | | | | |
| | − | + | − | + | − | + |
| Particle | 199 ± 9 | 179 ± 2 | 184 ± 2 | 180 ± 6 | 181 ± 8 | 193 ± 7 |
| Diameter (nm) | | | | | | |
| zeta-potential | 7 ± 4 | −15 ± 3 | 1 ± 5 | −17 ± 1 | −2 ± 1 | −18 ± 6 |
| (mV) | | | | | | |

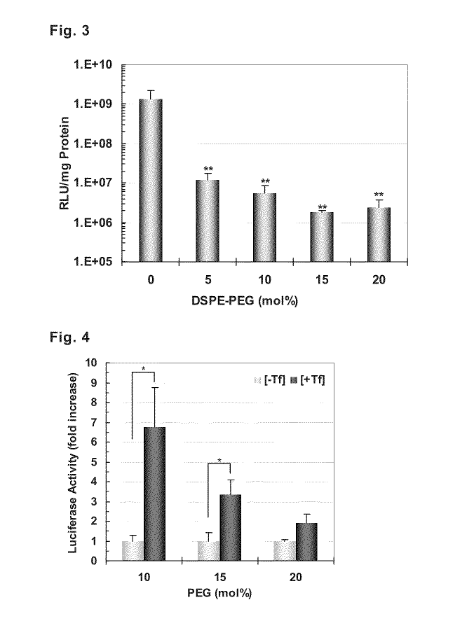

The gene expression activity observed after these pDNA core-encapsulated liposomes were transfected into the HeLa cells is shown in FIG. 4, which is based on the value obtained for Tf-unmodified liposomes as a control. At PEG concentrations of 10% and 15%, the gene expression activity was significantly increased 6.8 times and 3.3 times, respectively, by modification with Tf, and at a concentration of 20%, the cellular uptake inhibition effect of PEG was significant, and thus the increase of the activity was not significant. These results demonstrated that when liposomes were modified by using octaarginine as a cell membrane permeable peptide and binding Tf to PEG as a target cell-selective ligand, they were able to synergistically increase gene expression activity without being influenced by the cellular uptake inhibition by the modification with PEG.

### Example 2

### (1) Materials and Methods

### (a) Preparation of Liposomes Using Peptide Ligand

A ligand peptide having a cysteine residue (C) including thiol group at the end (CYGGRGNG) and a PEG-lipid derivative having maleimido group at the end, Mal-PEG-DSPE, were mixed at a molar ratio of 1:1, and the mixture was shaken at room temperature for 24 hours to obtain a peptide-bound PEG-lipid derivative (Pep-PEG-DSPE). Liposomes were prepared by mixing EPC and Chol at a molar ratio of 7:3, and adding 1 mol % of Rho-DOPE, and added with necessary amounts of Pep-PEG-DSPE and STR-R4 according to the post-modification method to prepare liposomes modified with peptide-bound PEG and R4 (dual-ligand type liposomes).

First, lipid solutions (ethanol solutions of EPC and Chol, and chloroform solution of Rho-DOPE) were put into a glass test tube in a total amount of 600 μmol/600 μL, and added with an equal volume of chloroform, the mixture was stirred, and then the solvent was evaporated under a nitrogen gas atmosphere or under reduced pressure. The resulting lipid films were added with PBS so that the lipid concentration became 8.0 mM, hydrated at room temperature for 10 minutes, and stirred for about 1 minute with a vortex mixer. The resulting liposome solution was passed through a membrane filter having a pore size of 400 nm 7 times by using an extruder to obtain liposomes having uniform particle sizes as large size liposomes. Small size liposomes were prepared by passing the large size liposome solution through a membrane filter having a pore size of 50 nm further 11 times using an extruder to obtain liposomes having uniform particle sizes.

Concentrations of Chol contained in the lipid membranes of the large size and small size liposomes made to have uniform particle sizes were quantified by using Cholesterol E-Test Wako to calculate total lipid concentrations. On the basis of the calculated total lipid amounts, amounts of STR-R4 and PEG-DSPE, or Pep-PEG-DSPE to be added were calculated, and they were added in the calculated amounts to the liposome solution to modify the liposomes. A necessary volume of an STR-R4 aqueous solution was added to the liposome solution, the mixture was shaken at 55° C. for 30 minutes to modify the liposomes with STR-R4, then the necessary amount of PEG-DSPE or Pep-PEG-DSPE was added, and the mixture was further shaken at 55° C. for minutes to modify the liposomes with PEG. The particle size and zeta potential were measured by the dynamic light scattering (DLS) method.

### 2) Evaluation of Property of Dual-Ligand Type Liposomes for Targeting Tumor Vascular Endothelial Cells

OSRC-II cells (cells derived from human renal cell carcinoma) were subcutaneously transplanted to BALB/cAJcl mice (4 weeks old, male, Clea Japan) on their left back to prepare cancer-bearing model mice. The cancer-bearing model mice of which tumor had grown to a tumor volume of 80 to 120 mm³ were administered with the fluorescence-labeled liposomes from the caudal vein under diethyl ether anesthesia (administration volume: 200 μL/mouse). After 24 hours, carcinoma tissues were collected under diethyl ether anesthesia. The collected carcinoma tissues were washed with PBS(−), added to PBS(−) added with a staining solution beforehand, and left standing at room temperature for 1 hour with shielding light. The staining solution was prepared by adding Hoechst 33342 (final concentration: 40 μM) and Isolectin-Alexa647 (final concentration: 20 μg/mL) to PBS(−). After the stained carcinoma tissues were washed with PBS(−), localization of the administered fluorescence-labeled liposomes in the carcinoma tissues was observed by using a confocal laser scanning microscope (Nikon Al).

### 3) Preparation of Doxorubicin-Encapsulated Dual-Ligand Type Liposomes

Liposomes were prepared by mixing hydrogenated soy phosphatidylcholine (henceforth abbreviated as “HSPC”) and Chol at a molar ratio of 7:3, and doxorubicin was encapsulated in the liposomes by the pH gradient method. Then, by adding necessary amounts of PEG-DSPE or Pep-PEG-DSPE, and STR-R4 according to the post-modification method, doxorubicin (Dox)-encapsulated dual-ligand type liposomes were prepared.

First, lipid solutions (ethanol solutions of HSPC and Chol) were put into a glass test tube in a total amount of 600 mol/600 μL, and added with an equal volume of chloroform, the mixture was stirred, and then the solvent was evaporated under a nitrogen gas atmosphere or under reduced pressure. The resulting lipid films were added with an ammonium sulfate solution (155 mM, pH 5.5) so that the lipid concentration became 20.0 mM, hydrated by warming at 65° C. for 10 minutes, and stirred for about 1 minute with a vortex mixer. The resulting liposome solution was passed through a membrane filter having a pore size of 400 nm 7 times by using an extruder warmed to 60° C. beforehand to obtain liposomes having uniform particle sizes as large size liposomes. The liposome solution was applied to a gel filtration column (solvent: PBS(−)) prepared by using Sephadex (registered trademark) G-25 Fine to replace the ammonium sulfate solution as the external aqueous phase with PBS(−). The total lipid concentration contained in the liposome solution was calculated by quantifying cholesterol, an appropriate amount of a Dox solution (3 mg/mL in PBS(−)) was added, and the mixture was stirred and shaken at 60° C. for 1 hour to enclose doxorubicin in the internal aqueous phases of the liposomes.

A necessary volume of an STR-R4 aqueous solution was added to the liposome solution, the mixture was shaken at 55° C. for 30 minutes to modify the liposomes with STR-R4, then the necessary amount of PEG-DSPE or Pep-PEG-DSPE was added, and the mixture was further shaken at 55° C. for 30 minutes to modify the liposomes with PEG. The particle size and zeta potential were measured by the dynamic light scattering (DLS) method.

In order to remove free doxorubicin not encapsulated in the liposomes, the liposome solution was subjected to ultrafiltration at 26° C. and 1000×g for 1 hour (Amicon Centrifugal Filter Units). The concentration of doxorubicin encapsulated in the liposomes was determined by measuring fluorescence intensity. A doxorubicin solution (3 mg/mL in PBS(−)) was serially diluted with methanol to concentrations of 0.0006, 0.0015, 0.006, and 0.015 mg/mL, and a calibration curve was created. The doxorubicin-encapsulated liposomes (10 μL) were added with methanol (990 μL) to dilute the liposomes. Fluorescence intensity of each sample was measured (Ex./Em.=450/590 nm), and concentration of doxorubicin encapsulated in the liposomes was determined by using the calibration curve.

### 4) Preparation of Doxorubicin-Encapsulated PEG Liposomes (Doxil)

Doxil was prepared according to the method reported in Mol. Pharm., 6, pp. 246-254, 2009. Liposomes were prepared by mixing HSPC, Chol, and PEG-DSPE at a molar ratio 3:2:0.265, and then doxorubicin was encapsulated by the pH gradient method. First, lipid solutions (ethanol solutions of HSPC, Chol and PEG-DSPE) were put into a glass test tube in a total amount of 600 mol/600 μL, and added with an equal volume of chloroform, the mixture was stirred, and then the solvent was evaporated under a nitrogen gas atmosphere or under reduced pressure. The resulting lipid films were added with an ammonium sulfate solution (155 mM, pH 5.5) so that the lipid concentration became 20.0 mM, hydrated at room temperature for 10 minutes, then ultrasonicated on a bath type sonicator, and further ultrasonicated with a probe type sonicator to prepare SUV liposomes. The liposome solution was subjected to gel filtration using Sephadex (registered trademark) G-25 Fine to replace the ammonium sulfate solution as the external aqueous phase with PBS(−). The total lipid concentration contained in the liposome solution was calculated by quantifying cholesterol, an appropriate amount of a Dox solution (3 mg/mL in PBS(−)) was added, and the mixture was stirred and shaken at 60° C. for 1 hour to enclose doxorubicin in the internal aqueous phases of the liposomes. After doxorubicin was encapsulated, in order to remove free doxorubicin not encapsulated in the liposomes, the liposome solution was subjected to ultrafiltration at 26° C. and 1000×g for 1 hour (Amicon Centrifugal Filter Units). A doxorubicin solution (3 mg/mL in PBS(−)) was serially diluted with methanol to concentrations of 0.0006, 0.0015, 0.006, and 0.015 mg/mL, and a calibration curve was created. The doxorubicin-encapsulated liposomes (10 μL) were added with methanol (990 μL) to dilute the liposomes. Fluorescence intensity of each sample was measured (Ex./Em.=450/590 nm), and concentration of doxorubicin encapsulated in the liposomes was determined by using the calibration curve.

### 5) Investigation of Antitumor Effect Obtainable by Using Dual-Ligand Type Liposomes

OSRC-II cells (cells derived from human renal cell cancer) were subcutaneously transplanted to BALB/cAJcl mice (4 weeks old, male, Clea Japan) on their left back to prepare cancer-bearing model mice. The cancer-bearing model mice of which tumor grew to a tumor volume of 80 to 120 mm³ were administered twice (Day 0 and Day 3) with the prepared doxorubicin-encapsulated liposomes from the caudal vein under diethyl ether anesthesia (administration volume: 200 μL/mouse). After the administration, the tumor volume was periodically measured.

Method for calculating tumor volume: Volume=Major axis×(Minor axis)²×0.5

TABLE 4

| | | | | |
|---|---|---|---|---|
| Formulation | PEG-LP | NGR-PEG-LP | R4/PEG-LP | Dual-LP |

Large size

| | | | | |
|---|---|---|---|---|
| Diameter (nm) | 286 ± 30 | 287 ± 14 | 279 ± 14 | 305 ± 23 |
| zeta-potential | −9.2 ± 1.9 | −13.4 ± 2.5 | −7.0 ± 1.3 | −9.2 ± 2.8 |
| (mV) | | | | |

Amount of PEG-lipid and R4

| | | | | |
|---|---|---|---|---|
| PEG-DSPE | 10 mol % | — | 10 mol % | — |
| NGR-PEG-DSPE | — | 10 mol % | — | 10 mol % |
| STR-R4 | — | — | 2.5 mol % | 2.5 mol % |

*Fluorescence-labeled liposomes and doxorubicin-encapsulated liposomes had the same composition and substantially the same physical properties.

TABLE 5

| | | |
|---|---|---|
| Formulation | PEG-LP | Doxil |

Small size

| | | |
|---|---|---|
| Diameter (nm) | 91 ± 3 | 88 ± 6 |
| zeta-potential (mV) | −9.1 ± 4.4 | −10.3 ± 0.2 |
| | *Fluorescence-labeled | * Doxorubicin- |
| | liposomes | encapsulated liposomes |

Amount of PEG-lipid and R4

| | | |
|---|---|---|
| PEG-DSPE | 10 mol % | 5.3 mol % |
| NGR-PEG-DSPE | — | — |
| STR-R4 | — | — |

### (2) Results

### 1) Antitumor Effect Obtained by Using Dual-Ligand Type Liposomes

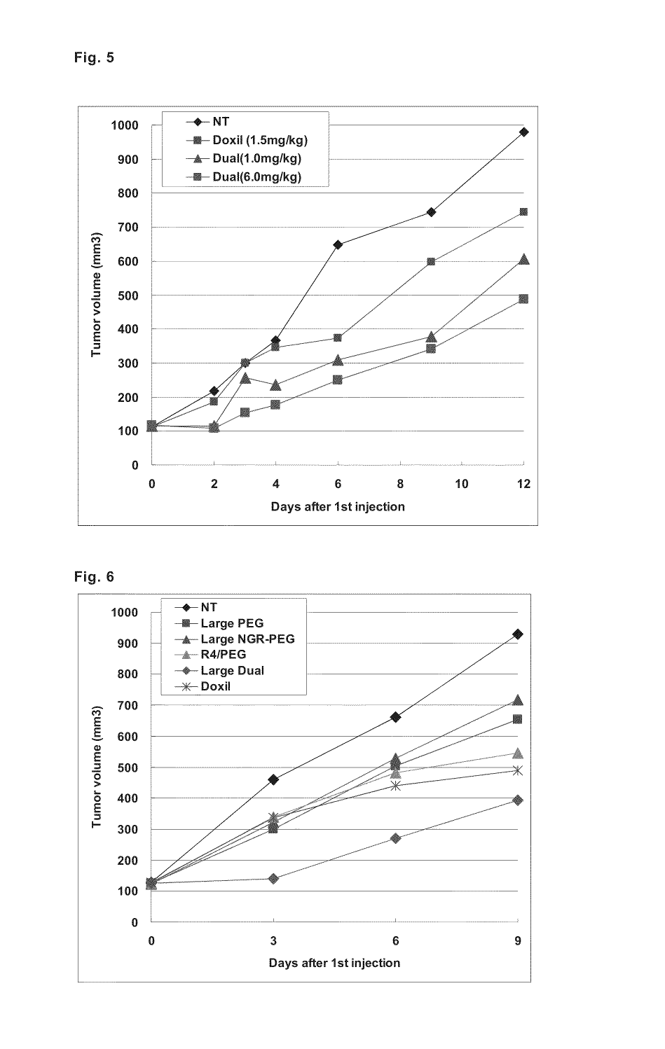

As for the tumor vascular endothelial cell-directive dual-ligand type liposomes encapsulating doxorubicin, the large size liposomes (about 300 nm) gave higher antitumor effect compared with the small size liposomes (100 nm). Further, the large size liposomes gave higher antitumor activity at a dose of 6.0 mg/kg compared with that obtained with a dose of 1.0 mg/kg (FIG. 5). The large size liposomes (dose: 6.0 mg/kg) gave antitumor action higher than that of Doxil (particle size: 100 nm) clinically applied. Further, the dual-ligand type liposome modified with the ligand-bound PEG and R4 gave stronger antitumor activity compared with the liposomes modified only with PEG or only with the ligand-bound PEG, or the liposomes modified with PEG and R4 (all were large size liposomes) (FIG. 6).

### 2) Evaluation of Property of Dual-Ligand Type Liposomes for Targeting Tumor Vascular Endothelial Cells

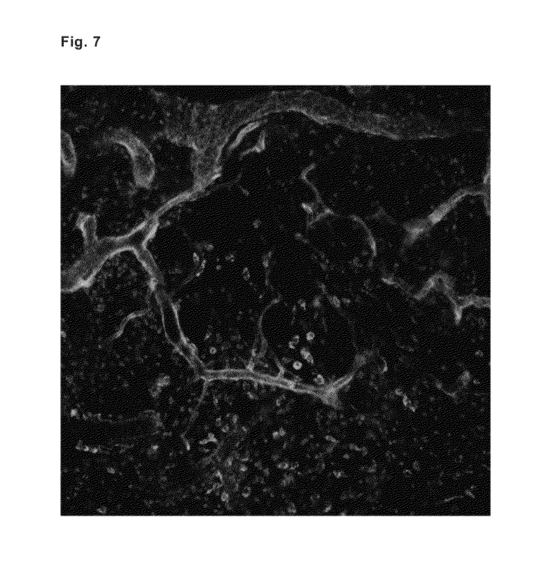

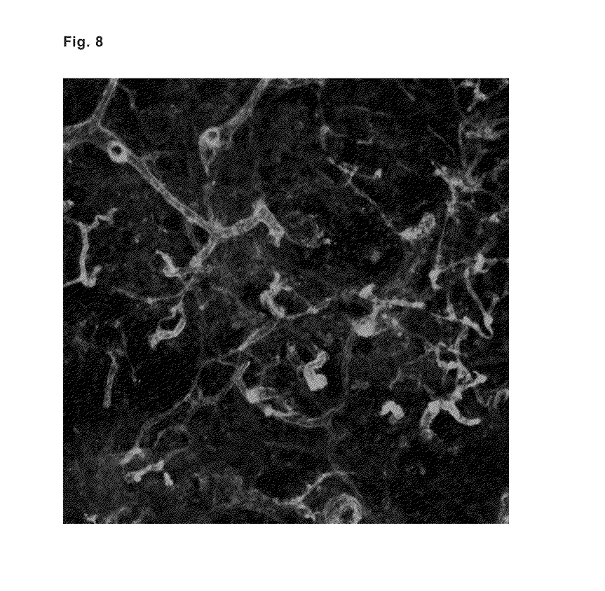

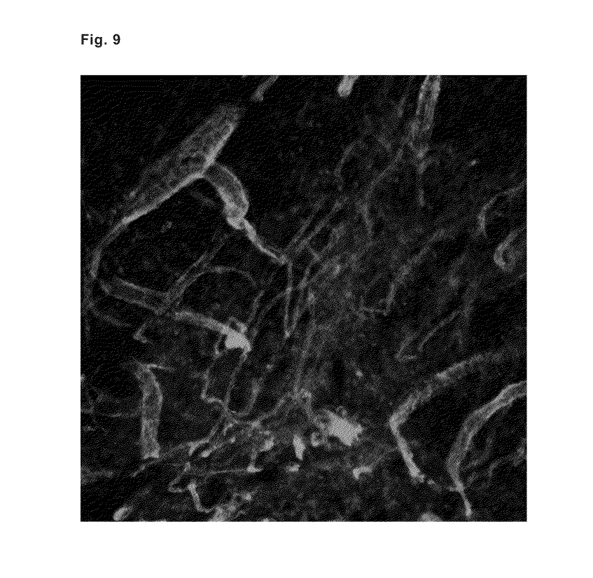

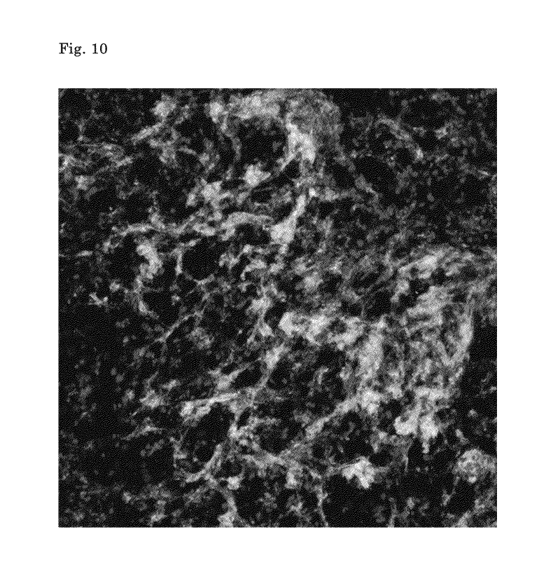

With the liposomes modified only with PEG, or the liposomes modified with PEG and R4, almost no red signal indicating presence of the liposomes was observed in the tumor vascular endothelial cells (FIGS. 7 and 9, respectively). Further, with the liposomes modified with the ligand peptide-bound PEG, although slight increase of the signal was observed, substantial change was not observed (FIG. 8). Whilst, with the dual type liposomes modified with the ligand-bound PEG and R4, presence of many signals was observed in the tumor vascular endothelial cells (FIG. 10). On the basis of these results, it was demonstrated that the liposome of the present invention had high target permeability for tumor vascular endothelial cells.

### INDUSTRIAL APPLICABILITY

The lipid membrane structure of the present invention simultaneously achieves superior in vivo stability, selectivity for target cell provided by a ligand, and cell permeability, and therefore, the structure is extremely useful for uses as, for example, a lipid membrane structure for delivering a nucleic acid containing a gene into a cell and expressing the gene, or a lipid membrane structure for selectively delivering an antitumor agent to a malignant tumor.

What is claimed is:

1. A lipid membrane structure for delivering a substance to a target cell, wherein lipid membrane is modified with the following (a) and (b):
(a) a polyalkylene glycol bound with a target cell-selective ligand; and
(b) a polypeptide comprising a plurality of arginine residues.

2. The lipid membrane structure according to claim 1, wherein the lipid membrane structure is a liposome.

3. The lipid membrane structure according to claim 1, wherein surface of the lipid membrane structure is modified with (a) the polyalkylene glycol and (b) the polypeptide.

4. The lipid membrane structure according to claim 1, wherein the target cell-selective ligand is a ligand that can specifically bind to a receptor expressed outside a cell membrane of the target cell.

5. The lipid membrane structure according to claim 1, wherein the target cell-selective ligand is bound to a tip end portion of (a) the polyalkylene glycol.

6. The lipid membrane structure according to claim 1, wherein (a) the polyalkylene glycol and (b) the polypeptide are modified with a hydrophobic group, and the hydrophobic group is inserted into the lipid membrane.

7. The lipid membrane structure according to claim 1, wherein (b) the polypeptide is a polypeptide containing 4 to 20 contiguous arginine (SEQ ID NO: 3).

8. The lipid membrane structure according to claim 1, wherein (a) the polyalkylene glycol is a polyethylene glycol.

9. The lipid membrane structure according to claim 1, wherein the substance to be delivered is encapsulated in the inside of the lipid membrane structure.

10. The lipid membrane structure according to claim 9, wherein a nucleic acid containing a gene and a cationic polymer are encapsulated in the inside of the lipid membrane structure.

11. The lipid membrane structure according to claim 9, wherein an antitumor agent is encapsulated in the inside of the lipid membrane structure.

12. The lipid membrane structure according to claim 11, wherein the antitumor agent is doxorubicin.

13. The lipid membrane structure according to claim 11, wherein the target cell-selective ligand is a ligand peptide.

14. The lipid membrane structure according to claim 11, which is in the form of a liposome.

15. The lipid membrane structure according to claim 14, which has a particle size in the range of about 200 nm to 400 nm.

16. A pharmaceutical composition containing the lipid membrane structure according to claim 9 as an active ingredient.


SEQUENCE LISTING (4 sequences)

SEQ ID NO: 1 - protein, 8 aa, Artificial Sequence
  Description of Artificial Sequence Synthetic peptide
CYGGRGNG

SEQ ID NO: 2 - protein, 8 aa, Artificial Sequence
  Description of Artificial Sequence Synthetic peptide
RRRRRRRR

SEQ ID NO: 3 - protein, 20 aa, Artificial Sequence
  Description of Artificial Sequence Synthetic peptide
RRRRRRRRRRRRRRRRRRRR

SEQ ID NO: 4 - protein, 4 aa, Artificial Sequence
  Description of Artificial Sequence Synthetic peptide
RRRR